(19) United States
(12) Patent Application Publication
(10) Pub. No.: US 2019/0045780 A1
(43) Pub. Date: Feb. 14, 2019

(54) DISINFECTANT AGENT

(71) Applicant: Dantor Biotech AG, Hunenberg (CH)

(72) Inventor: Sven Reichwagen, Ahlen (DE)

(73) Assignee: Dantor Biotech AG, Hunenberg (CH)

(21) Appl. No.: 16/077,660

(22) PCT Filed: Feb. 10, 2017
(86) PCT No.: PCT/EP2017/052980

§ 371 Date: Aug. 13, 2018

(30) Foreign Application Priority Data
Feb. 12, 2016 (DE) 10 2016 102 485.6

(51) Int. Cl. A01N 25/22; A01N 37/16; A01N 25/04; A61L 2/18; A61L 2/22

(52) U.S. Cl. CPC A01N 25/22; A01N 37/16; A61L 2/22; A61L 2/186; A01N 25/04

(57) ABSTRACT

The invention relates to an agent for the disinfection of surfaces and the destruction of bacterial metabolic products, said agent containing in aqueous solution at least one percarboxylic acid together with a polyvinylpyrrolidone as stabilizing agent.

The invention relates to an agent for the disinfection of surfaces and destruction of bacterial metabolic products, said agent containing at least one percarboxylic acid together with a stabilizing agent in aqueous solution.

Basically, the use of percarboxylic acids for disinfection purposes is basically known, as for example disclosed via EP 0 677 990 B1. In particular, it describes the use of peracetic acid. The disinfectant is suited, inter alia, for the treatment of medical equipment, for instance endoscopes.

Percarboxylic acid solutions are produced by converting carboxylic acid with hydrogen peroxide in an aqueous medium in the presence of a catalyst, usually sulfuric acid. The reaction that takes place results in an equilibrium forming between the percarboxylic acid as reaction product on the one hand and the reactants, i.e. the carboxylic acid and hydrogen peroxide on the other. After the reaction has been completed the reaction mixture is often diluted further through the application of a solvent to bring about the desired lower percarboxylic acid concentration. For purposes such as cleaning of toilets solutions may for example be employed that contain 0.5 to 1% w/w of peracetic acid. However, the percarboxylic acid is produced initially in the form of a higher concentrated solution because the time period required to adjust the equilibrium would otherwise be excessively long.

Even if a concentrated equilibrium solution of the percarboxylic acid is diluted with water, the system equilibrium point changes in favor of the regeneration of the original reactants; and such an equilibrium adjustment may as well require a long period of time to complete, i.e. in the range of several weeks or even months. Accordingly, the diluted percarboxylic solution is a solution of variable composition the effectiveness of which lessens more and more due to the hydrogen peroxide being subject to decomposition.

From WO 2006/125 657 A2, to whose content explicit reference is made, an aqueous disinfectant and sterilization agent is known, in which percarboxylic acids are stabilized by the addition of sorbitan esters. This method has proven to be successful basically. However, the use of sorbitan esters limits the range of application of such stabilized percarboxylic acids, for instance in the foodstuff and medical and care sectors.

Based on this state of the art, it is the objective of the invention to provide an alternative stabilizing agent for percarboxylic acids that enables the application of disinfectants stabilized with said agent in a variety of areas and, especially, also in the foodstuff industry and in the medical and care sectors. Such a stabilizing agent should physiologically be unobjectionable, well tolerated by the skin and stabilize percarboxylic acids sufficiently long on the treated surface to bring about a lasting effect.

This objective is achieved with an agent of the kind first mentioned above, which contains polyvinylpyrrolidone (PVP) as stabilizing agent.

The invention is based on findings according to which the addition of a significant quantity of polyvinylpyrrolidone considerably increases the stability of percarboxylic acids. It was found that the concentration of percarboxylic acid does not change over a long period of time, so that the agent can be used for months or years. The effect is attributed to the fact that polyvinylpyrrolidone complexes the peracid function and thus prevents the elimination of oxygen. At the same time, the aggressiveness of short-chain percarboxylic acids is reduced so that they can be used on the skin.

In addition, polyvinylpyrrolidone is a frequently used product introduced in the pharmaceutical and food industries, which is physiologically unobjectionable, well tolerated and, what is more, pleasant to the skin. In foodstuffs it is used as a binding and production agent, in tablets as a binding agent and for wound treatment purposes as a carrier substance for iodine.

Polyvinylpyrrolidone, also known as povidone and often abbreviated with PVP, is a highly water-soluble polymer with an amorphous structure. It has no melting point, but only a glass transition temperature, which ranges between 110 and 180° C., depending on the degree of polymerization. According to the invention, polyvinylpyrrolidones of all degrees of polymerization can be put to use.

Polyvinylpyrrolidones with an average molecular weight in the range of between 10,000 and 360,000 daltons are preferred. For example, polyvinylpyrrolidones having average molecular weights (weight average) of 15,000 daltons and especially 40,000 daltons (povidone K 30) have proven to be suitable.

For purposes in accordance with the invention, all percarboxylic acids that can be brought into aqueous solution can be used basically. These are for example saturated and unsaturated aliphatic as well as aromatic monocarboxylic acids having up to 12 carbon atoms, such as peracetic acid, perpropionic acid, persorbic acid and perbenzoic acid, hydroxycarboxylic acids with up to 8 carbon atoms, such as perlactic acid, percitric acid, permalic acid and pertartaric acid, as well as any mixtures thereof. Peracid may have several hydroxy and/or acid functions. In particular multifunctional percarboxylic acids, such as percitric acid, have proven to be very efficient for purposes in accordance with the invention.

The content of PVP-stabilized percarboxylic acid (PVP plus percarboxylic acid) in the inventive agent expediently amounts to between 0.1 and 50% w/w, preferably between 1.0 and 25% w/w. The weight ratio PVP to percarboxylic acid ranges between 1:10 and 10:1, preferably between 1:5 and 5:1.

Naturally, the higher the percarboxylic acid concentration the higher the effectiveness of the agent; for many applications, however, it will be sufficient to use concentrations of about 1% w/w. As a rule, a concentrate is prepared initially that has a percarboxylic acid concentration in the range of between 10 and 20% w/w but said concentration will subsequently be reduced through dilution using water or a solvent.

It has proven particularly advantageous to introduce into the agent at least two percarboxylic acids because experience has shown that this may often produce synergistic effects. It was thus found that different percarboxylic acids were particularly suited for different specific purposes. For example, a number of toxins, both microtoxins and endotoxins, having an aldehyde or ketonic function are effectively destroyed by means of perbenzoic acid via a Baeyer-Villiger oxidation. Perbenzoic acid can be used against aflatoxins and acts as a catalase inhibitor. On the other hand, peracetic acid is suited to effectively attack pore proteins of bacteria whereas percitric acid has proven especially suited to decompose biofilms. Persorbic acid is capable of entering lipid layers and in this manner penetrates through cell membranes in order to deactivate bacteria.

In addition to the percarboxylic acid stabilized with PVP in aqueous solution the inventive agent may contain a surfactant that is non-reactive with the percarboxylic acid in order to increase the wettability. In this respect, polysorbates in an amount of 0.5 to 5% w/w, such as Tween (polysorbate 20) or sodium dodecyl sulfate, are preferred.

The inventive agent may contain common additives, in particular solvents, sequestering agents, pH regulators, corrosion inhibitors, other peracid stabilizers, complexing agents, defoaming agents, colorants and odorants, thickeners and care products. It goes without saying that all these additives must be stable with respect to percarboxylic acids and also do not promote the decay of percarboxylic acids. Corrosion inhibitors are especially useful if the composition is to be used for the treatment of metal surfaces. For example, alkali metal phosphates, preferably potassium phosphates, such as dipotassium hydrogen orthophosphate, can be used as corrosion inhibitors.

The main solvent used is water. Moreover, the agent may contain organic solvents, in particular ethanol and isopropanol. Alcohol offers advantages in that it has additional bactericidal and fungicidal properties so that the germicidal effects of the inventive composition is even enhanced.

For the application as disinfectant of body surfaces, i.e. skin and mucous membrane, the product is preferably formulated in the form of a spray, gel, cream or lotion. Here, too, it is mandatory that the formulation additives offer oxidation stability.

The pH value of the aqueous solution should be in the acidic range. Percarboxylic acids tend to decompose in an alkaline environment. Typically, the pH of the diluted solution for application purposes ranges between 0 and 7, especially between 3 and 6. If necessary, the pH value can be adjusted by using pH regulators.

The agent proposed by the invention has proven to be highly effective against bacterial, viral and fungal infections. It is also suitable for destroying metabolic products of microorganisms. This applies both to toxic metabolic products (biotoxins) of bacteria as well as to adhesins, i.e. bacterial products that promote the adhesion and the growth of bacteria on the body or on wounds. Accordingly, the agent according to the invention is suitable as a disinfectant and cleanser for the skin and mucous membrane.

In the fight against microorganisms, the release of oxygen radicals causes rapid oxidation of the outer shell/cell membrane. Percarboxylic acids also attack and damage enzymes and other proteins as well as nucleic acids. The resulting blockage of metabolic pathways (energy metabolism, protein biosynthesis) rapidly leads to cell death. Aside from this, the inventive composition will also destroy biotoxins—aflatoxins, endotoxins, mykotoxins and allergens.

The percarboxylic acids of the agent proposed by the present invention are capable of modifying the tertiary structure of proteins in such a way that these lose their properties. Thus disulfide bridges are broken up by oxidation and thiol groups are oxidized to form sulfonic acids.

The compositions according to the invention are also capable of deactivating the microorganisms' centers of metabolism by oxidation of enzymes that primarily contain heavy metals for example copper. Viruses can be deactivated by causing the capsid proteins and envelope proteins to be oxidized. This leads to a disintegration of the nucleocapsid or to a modification of the capsid surface which results in the virus to become inactive.

Through oxidation and the destruction of harmful microorganisms associated with it, the percarboxylic acids reconvert into the carboxylic acid itself, so that ultimately only comparatively harmless products are produced.

The inventive agent may advantageously used for agriculture applications, for example in plant/crop protection, plant strengthening, for disinfecting and cleaning stables, for treating exhaust air and for disinfecting agricultural storage spaces and rooms. In this context, explicit reference is made to the applications mentioned in WO 2006/125 657 A2.

The agent proposed by the invention can also be used for the cleaning and disinfection of wounds, as well as for the treatment of foot and toenail fungus, against warts and as a washing-up liquid for oral and dental hygiene. It can also be used with animals for cleaning and disinfection as well as for the treatment of skin, fur/hide and claw/hoof diseases.

The inventive agent can be produced analogous to the principles described in WO 2006/125 657 A2. In addition to the respective carboxylic acids, their anhydrides and usual salts can also be used as starting materials. The following examples explain the procedure.

### EXAMPLE 1

In 90 g of a hydrogen peroxide solution (20%) in water, 5 g of citric acid are initially converted to obtain percitric acid in the presence of a catalytic amount of phosphoric acid at room temperature. After equilibrium adjustment, 5 g of PVP K30 are added to the mixture. This mixture is then diluted with ten times the amount of water to obtain application strength.

The equilibrium adjustment of the mixture may take several days. An excess of hydrogen peroxide shifts the equilibrium towards percarboxylic acid. For acceleration purposes, a small amount of a strong acid, for example methanesulfonic acid, phosphoric acid or sulfuric acid, can be added to the mixture as a catalyst.

The mixture obtained in this way is suitable for the treatment and disinfection of skin wounds. With a view to producing a gel, a common thickener, such as an acrylate, can be added.

### EXAMPLE 2

An active agent against warts is produced as follows:

90 g of a hydrogen peroxide solution in water (35%) are prepared, with 1 g of benzoic acid, 3 g citric acid and 1 g sodium diacetate being added at room temperature. The mixture is then stirred until the reaction equilibrium is obtained and following this 5 g of PVP K30 are added. After further stirring, 900 ml of deionized water is added, then another 20 g of PVP K30. Further dilution with 1000 ml deionized water results in the finished agent.

1. Agent for the disinfection of surfaces and the destruction of bacterial metabolic products, said agent containing at least one percarboxylic acid together with a stabilizing agent in aqueous solution, characterized in that polyvinylpyrrolidone (PVP) is used as stabilizing agent.

2. Agent according to claim 1, characterized in that the PVP has an average molecular weight ranging between 10,000 and 360,000 daltons.

3. Agent according to claim 2, characterized in that the PVP has an average molecular weight of 40,000 daltons.

4. Agent according to claim 1, characterized in that the percarboxylic acid is a monocarboxylic acid, in particular benzoic acid.

5. Agent according to claim 1, characterized in that the percarboxylic acid is a hydroxypercarboxylic acid.

6. Agent according to claim 5, characterized in that the percarboxylic acid is perlactic acid, percitric acid, permalic acid or pertartaric acid.

7. Agent according to claim 1, characterized in that the weight ratio of percarboxylic acid with respect to PVP is in the range between 1:10 and 10:1.

8. Agent according to claim 1, characterized in that the aqueous solution contains 0.1 to 50% w/w, preferably 1 to 25% w/w, of percarboxylic acid stabilized with PVP.

9. Agent according to claim 1, characterized in that it also contains an oxidation-stable surfactant.

10. Agent according to claim 9, characterized in that it contains 0.5 to 5% w/w of polysorbate.

11. Agent according to claim 1, characterized in that the agent contains solvents, sequestrants, pH regulators, corrosion inhibitors, stabilizing agents for peracids, complexing agents, antifoaming agents, colorants and/or odorants.

12. Agent according to claim 1, characterized in that said agent has a pH value of 0 to 7, preferably 3 to 6.

13. Use of the agent in accordance with claim 1 for the sterilization of surfaces.

14. Use of the agent according to claim 1 for the destruction of bacterial metabolic products.

15. Use of the agent according to claim 1 for the disinfection of stables and for plant/crop protection purposes.

16. Composition in the form of a spray or gel containing the agent according to claim 1.

17. Use of polyvinylpyrrolidone for stabilizing percarboxylic acids in aqueous solution.
